(19) United States
(12) Patent Application Publication
(10) Pub. No.: US 2019/0352247 A1
(43) Pub. Date: Nov. 21, 2019

(54) Two-Phase Reactions in Microdroplets

(71) Applicant: The Board of Trustees of the Leland Stanford Junior University, Stanford, CA (US)

(72) Inventors: Richard N. Zare, Stanford, CA (US); Xin Yan, College Station, TX (US)

(21) Appl. No.: 16/479,024

(22) PCT Filed: Feb. 8, 2018
(86) PCT No.: PCT/US2018/017428

§ 371 Date: Jul. 18, 2019

Related U.S. Application Data
(60) Provisional application No. 62/457,757, filed on Feb. 10, 2017.

(51) Int. Cl. C07C 51/255; B01F 5/02; B01F 3/08; B01F 3/04; B01J 14/00; B01J 10/00; B05B 7/08

(52) U.S. Cl. CPC C07C 51/255; B01F 5/0256; B01F 3/0803; B01F 3/0865; B01F 2215/0036; B01J 14/00; B01J 10/00; B05B 7/0846; B01F 3/04049

(57) ABSTRACT

Improved two phase chemical reactions (liquid-liquid or liquid-gas) are provided by forming microdroplets of either or both liquid reagents and configuring the reaction as a collision between the microdroplet reagent and the other reagent. We have found that this approach can provide high reaction yields in short times (<1 s) without the use of a phase transfer catalyst.

### FIELD OF THE INVENTION

This invention relates to two-phase chemical reactions in microdroplets.

### BACKGROUND

Organic reactions in systems containing two immiscible phases (either liquid-liquid or gas-liquid) appear in a number of important applications. Reactions between two substances located in different phases of a mixture are often inhibited because of the inability of reagents to come together. Phase-transfer catalysis (PTC) is commonly used to enhance reaction rates, making feasible a wide variety of synthetic reactions not possible in a single phase. However, phase transfer catalysis has its own associated issues, such as cost, thermal instability, and especially separation/recycling of catalysts. Accordingly, it would be an advance in the art to carry out two-phase liquid-liquid or liquid-gas chemical reactions on a preparative scale without using phase transfer catalysts with high yield and in a short time.

### SUMMARY

In this work, we provide a strategy to perform superfast two-phase reactions (both liquid-liquid and liquid-gas reactions) in microdroplets without using a phase transfer catalyst. By using microdroplets for liquid phases, interfacial area between the two phases can be increased by many orders of magnitude.

Numerous applications are possible. This process can be used for nearly all industrial processes that employ liquid-liquid of gas-liquid two-phase synthesis. Such reactions in pharmaceutical processes and polymer syntheses include but are not limited to C-, N-, O- and S-alkylation, etherification, esterification, transesterification, condensation, carbene reaction, nucleophilic displacement epoxidation, oxidation and polymerization. For reviews of two-phase chemical reactions that use phase transfer catalysts, see “Phase-Transfer Catalysis, Marc Halpern, Ullmann's Encyclopedia of Industrial Chemistry, 2000, 26, 495-500.” and “Phase transfer catalysis in pharmaceutical industry -where are we? Fedorynski, M., Jezierska-Zieba, J., Kakol B. Acta Poloniae Pharmaceutica—Drug Research, 2008, 65, 647- 654,” both of which are hereby incorporated by reference in their entirety.

Significant advantages are provided. This methodology avoids using a phase transfer catalyst but still enables the two-phase reaction to occur within milliseconds in high yield. Moreover, it is expected that this process can be scaled for industrial production. All the disadvantages from using a phase transfer catalyst such as cost, thermal instability, and especially separation/recycling of catalysts can be avoided. Moreover, the time required for the reaction to occur can be greatly reduced. Typical phase transfer catalysis happens within minutes to days, whereas the method we are describing is less than a second. Our method also greatly simplifies the work-up of the product. PTC for anions are often quaternary ammonium salts (Q+). The recovery is usually by aqueous extraction of the organic layer and re-extraction with an appropriate solvent. Removing the last traces of Q+, usually by ion-exchange, can be difficult and expensive but is often required for drugs and Q+ sensitive products.

Variations and modifications include varying process parameters including but not limited to: flow rates of the two immiscible liquids, sheath gas/nebulization gas pressure, capillary diameter, surface materials, reagent concentration, active assistance such as temperature, sonication, electric field and radiation. It is also expected that configurations other than colliding microdroplets may also be effective. For example, microdroplets of one reagent could collide with a thin film of another reagent.

### BRIEF DESCRIPTION OF THE DRAWINGS

FIGS. 1A-B show two embodiments of the invention.

FIGS. 2A-B show two further embodiments of the invention.

FIG. 3 shows a reaction scheme for the experiment of section B.

FIG. 4A shows an experimental arrangement for the experiment of section B.

FIG. 4B is a gas chromatography spectrum relating to the arrangement of FIG. 4A.

FIG. 4C is a gas chromatography spectrum relating to control experiments for comparison to the result of FIG. 4B.

FIGS. 5A-F show various further experimental arrangements for the experiment of section B.

FIG. 5G shows gas chromatography spectra relating to the arrangements of FIGS. 5A-F.

FIG. 6 is a table of results for the experiment of section B.

FIG. 7 shows a scaled-up experimental arrangement for the experiment of section B.

FIGS. 8A-C are gas chromatography spectra relating to various collection substrates for the experiment of section B.

FIGS. 9A-D show yield as a function of several parameters of the experiment of section B.

FIGS. 10A-B show two experimental arrangements for the experiment of section C.

FIG. 11 shows a reaction scheme for the experiment of section C.

FIG. 12 shows conversion % for various configurations of the experiment of section C.

FIG. 13 shows conversion % vs. surface-area to volume ratio for the microdroplets of the experiment of section C.

FIG. 14 is a table of results for the experiment of section C.

FIGS. 15A-F show SEM images of meshes that were used in sprayers to provide microdroplets for the experiment of section C.

FIG. 16 shows yield for various configurations of the experiment of section C.

FIG. 17 shows an arrangement for collecting product of the experiment of section C.

FIG. 18 shows the effect of various catalysts on yield for the experiment of section C.

FIG. 19 shows the effect of various solvents on yield for the experiment of section C.

FIGS. 20A-C show several ways to decrease microdroplet size that were investigated as part of the experiment of section C.

### DETAILED DESCRIPTION

Section A provides a general overview of concepts relating to embodiments of the invention. Section B relates to an experimental demonstration of liquid-liquid reactions according to principles of the invention. Section C relates to an experimental demonstration of liquid-gas reactions according to principles of the invention.

### Section A) Overview

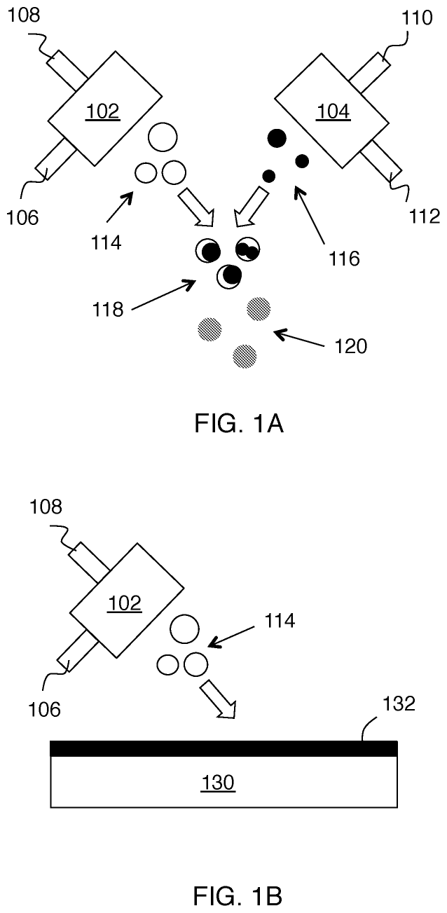

FIG. 1A shows a first embodiment of the invention. In this example, a first liquid reagent is nebulized in a first shearing gas flow to provide first microdroplets 114 of the first reagent. Here first microdroplets 114 are provided by a spray nozzle 102 having a gas inlet 106 and a liquid inlet 108. A second liquid reagent is nebulized in a second shearing gas flow to provide second microdroplets 116 of the second reagent. Here second microdroplets 116 are provided by a spray nozzle 104 having a liquid inlet 110 and a gas inlet 112. The first liquid reagent and the second liquid reagent are immiscible with respect to each other. Without being bound by theory, it is expected that this configuration can result in the microdroplets being forced together as schematically shown by 118.

Two substances are said to be immiscible if there are certain proportions in which the mixture of the two substances does not form a solution. Nebulizing is breaking up liquid solutions or suspensions into small aerosol droplets in a gas. Here an aerosol is a mixture of liquid particles in gas. Physical effects that can provide a nebulizing effect include ultrasonic vibrations and shearing gas flows, which are often said to be turbulent gas flows. Here microdroplets are defined as having a diameter in the range of 100 microns to 0.1 microns, and thin films are defined as having a thickness of 100 microns or less. Larger droplets or thicker films are not expected to provide good results, most likely because the required phase mixing does not occur in larger structures.

As shown on FIG. 1A, the first microdroplets are directed at the second reagent to provide a chemical reaction between the first and second liquid reagents by colliding the first microdroplets with the second reagent to form a product 120. FIG. 1B shows an alternative where the second liquid reagent is configured as a thin film 132 disposed on a substrate 130.

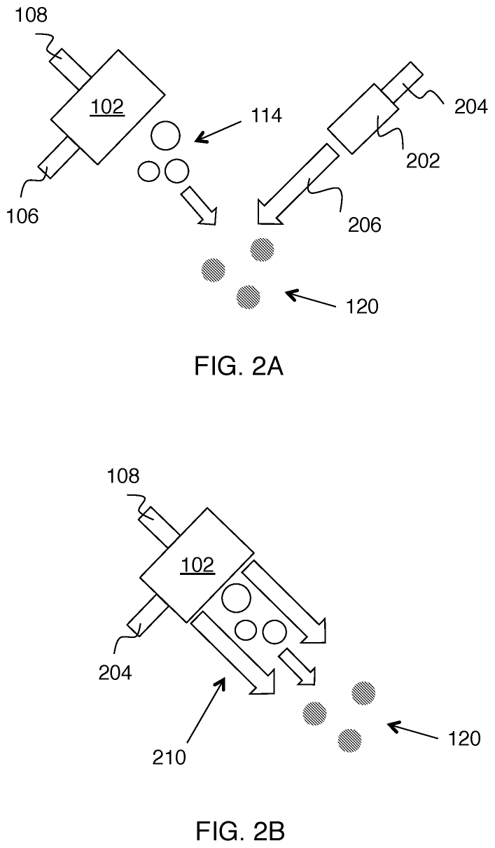

The present approach can also be used for gas-liquid reactions. FIG. 2A shows an example. Here a liquid reagent is nebulized in a first shearing gas flow to provide microdroplets 114 of the liquid reagent. As above, first microdroplets 114 are provided by a spray nozzle 102 having a gas inlet 106 and a liquid inlet 108. A gaseous reagent 206 is provided in this example by a nozzle 202 having a gas inlet 204. Microdroplets 114 are directed at the gaseous reagent to provide a chemical reaction between the liquid reagent and the gaseous reagent by colliding the first microdroplets with the gaseous reagent to form a product 120.

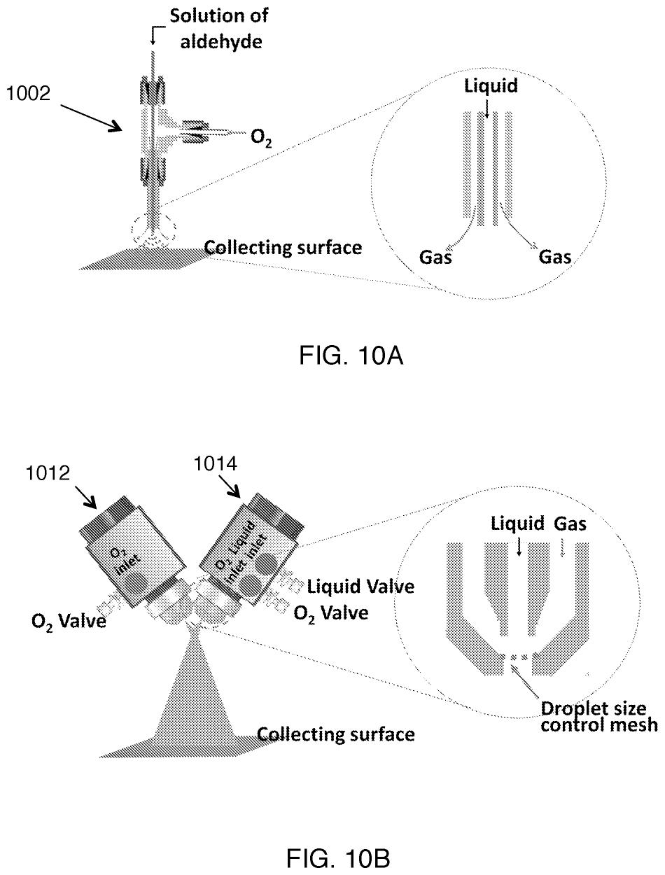

The example of FIG. 2A shows the gaseous reagent 206 being provided in a second gas flow that is distinct from the first shearing gas flow, where the first shearing gas flow and the second gas flow are directed at each other. FIG. 2B shows an alternative where the first shearing gas flow is a shearing gas flow of the gaseous reagent. Here gas inlet 204 admits the gaseous reagent to spray nozzle 102, and the resulting emission from spray nozzle 102 includes both reagents as schematically indicated by 210 on FIG. 2B (here the circles are microdroplets of the liquid reagent and the large block arrows depict the gaseous reagent). Product 120 is formed by the gaseous reagent reacting with the liquid microdroplets in the same gas flow. As indicated in more detail below (e.g. on FIG. 10B) the ideas of FIGS. 2A and 2B can be practiced in combination, where the first shearing gas flow of FIG. 2A is also a flow of the gaseous reagent.

In all cases, the chemical reaction can be selected from the group consisting of: C-, N-, O- and S-alkylation; etherification; esterification; transesterification; condensation; carbene reaction; nucleophilic displacement epoxidation; oxidation; and polymerization. Preferably a reaction time of the chemical reaction is 1 second or less.

### Section B) Liquid-liquid Reactions

Organic reactions in systems containing two immiscible liquid phases appear in a number of important applications in chemical, pharmaceutical, and polymer synthesis. The reaction between two substances located in different phases of a mixture is often inhibited because of the inability of reagents to come together. Traditionally, a phase-transfer catalyst (PTC) is used to enhance reaction rates, making feasible a wide range of synthetic reactions not possible in a single phase. The most common arrangement for PTC involves the transport of a water-soluble reactant into an immiscible organic solvent (Starks extraction mechanism) or the transport of a reactant at the interface of two immiscible solvents (Makosza interfacial mechanism) with an appropriate hydrophobic phase-transfer catalyst. Two-phase reactions are carried out between immiscible phases; thus, the nature of the interface and the physical properties of the reacting compounds at the interface become very important in promoting the desired reaction at a satisfactory rate. Methods that can enlarge the interfacial contact area between the two phases should effectively enable better mass transfer, resulting in better product conversion in reduced time. Available methods such as vigorous magnetic or mechanical stirring, ultrasonic irradiation and rotor-stator homogenizer accelerate two-phase reactions to some extent, but a phase-transfer catalyst is obligatorily needed in those methods.

However, one cannot avoid problems associated with phase-transfer catalysts, such as thermal instability, cost, and especially the need to separate and recycle catalysts. PTC for anions are often quaternary ammonium salts (Q⁺). The recovery is usually accomplished by extraction, distillation, adsorption, or binding to an insoluble support. Most methods employ an organic layer containing about 90% Q⁺, but the need exists to recycle at least ten times with no Q⁺ loss. Removing residual traces of Q⁺, usually by ion-exchange, can be difficult and expensive, but it is often required for synthesis of drugs and Q⁺-sensitive products. We present a methodology that avoids using a phase-transfer catalyst but still enables the two-phase reaction to occur within milliseconds in yields of 50-75%.

Recent studies have shown many single-phase reactions can be dramatically accelerated in microdroplets created by spray-based ionization, surface drop-casting, and microfluidics. Microdroplets as tiny reactors have a strikingly different reactive environment for reagents from that of the corresponding bulk phase. How exactly the reaction is accelerated in microdroplets however remains to be fully understood given both the size and time scales involved. Many factors are thought to contribute to the reaction acceleration such as microdroplet evaporation, confinement of reagents, alteration of pH of the microdroplet surface, and probably one of the most important features, high surface-to-volume ratio of the microdroplet. A reaction/adsorption model describing adsorption of molecules at interfaces in small droplets plays an important role in microdroplet accelerating reactions. Observation of an extra acceleration for p-methylbenzaldehyde in microdroplet reaction with 6-hydroxy-1-indanone by cooperative interactions between p-methylbenzaldehyde and p-nitrobenzaldehyde well supported the above model based on the assumption that more reagents stayed at the interface than in the body.

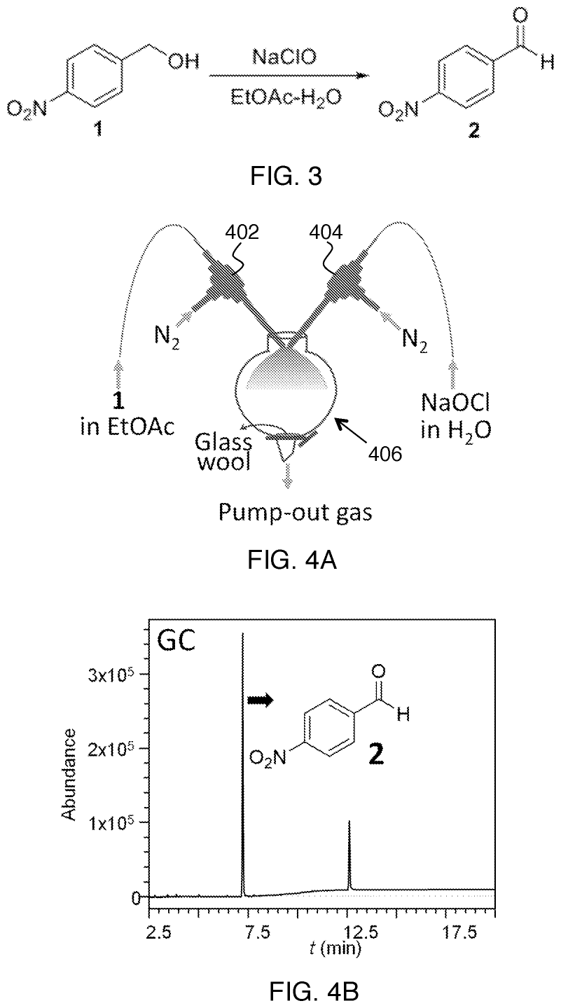

In this work, we provide a strategy to perform superfast two-phase reactions in microdroplets without using a phase-transfer catalyst. Bulk liquid-liquid system was dispersed as small aerosol droplets in a manner such that the interfacial area between the two phases is increased by many orders of magnitude. We also used the extreme case, reactions that only occur at the interface, to elucidate the important role of the microdroplet interface in two-phase reaction acceleration. Stevens oxidation without using a phase-transfer catalyst (Scheme 1 as shown on FIG. 3) was chosen as a proof of concept. Sodium hypochlorite (NaOCl) was used to oxidize 4-nitrobenzyl alcohol (1) to 4-nitrobenzaldehyde (2).

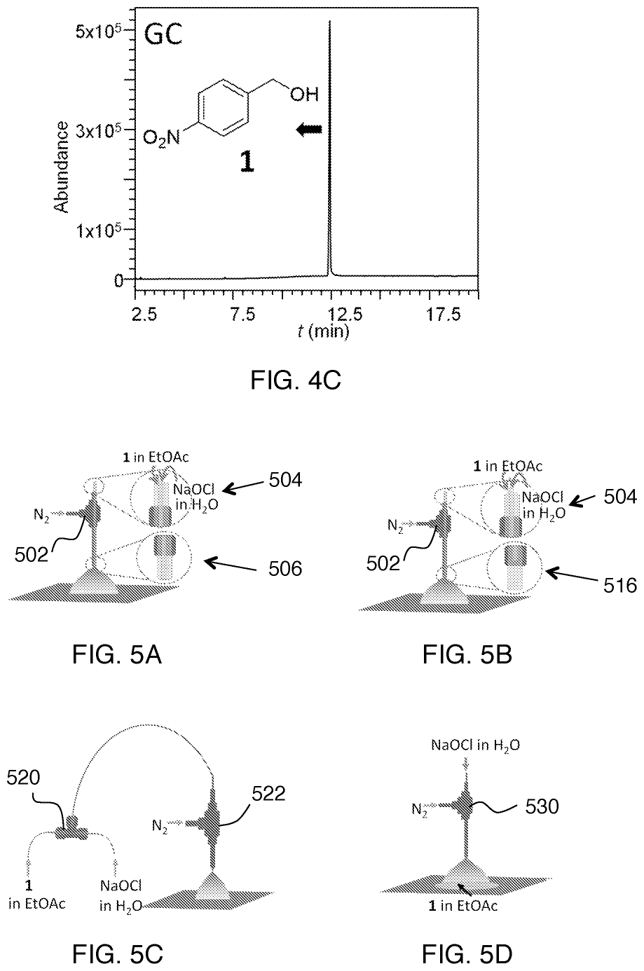

FIG. 4A shows a two-phase oxidation reaction between 4-nitrobenzyl alcohol 1 (0.2 M) in ethyl acetate (EtOAc) with NaOCl (12.5%) performed in microdroplets generated in chamber 406 by the atomization of respective bulk solutions with a turbulent nebulizing gas (dry N₂) at 120 psi in sprayers 402 and 404 respectively. Oxidation was initiated by the rapid mixing of droplets containing each reactant at the spray emitters and progressed as the microdroplets travelled in air. The resulting products in the merged plumes were collected using a glass separation funnel for 10 min. Exhaust gas was pumped out from the bottom, while glass wool was used to cover the gas outlet, avoiding loss of products. The distance and angle between the two microdroplet spray emitters influenced the formation of products, as described later.

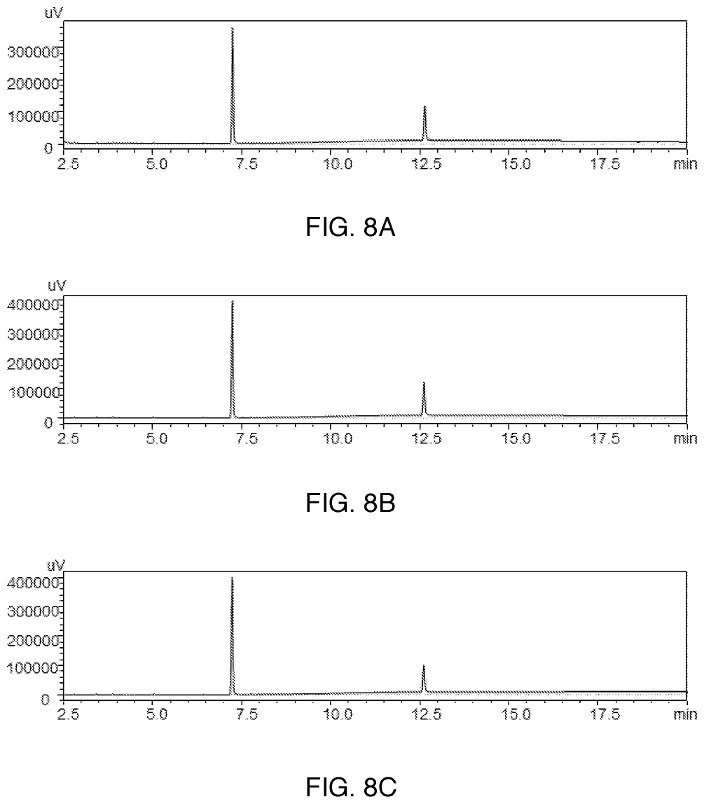

The reaction mixture was extracted with EtOAc and analyzed by gas chromatography (GC). FIG. 4B is the resulting gas chromatography (GC) spectrum that identifies the formation of the product 4-nitrobenzyladehyde (2) in 72% yield. Other materials such as aluminum foil and Teflon were also investigated as collection surfaces, with no apparent difference in product formation (FIGS. 8A-C). More specifically, FIGS. 8A-C show GC of two-phase microdroplet oxidation reaction between 4-nitrobenzyl alcohol (1) with NaOCl to form 4-nitrobenzaldehyde (2) collected on (FIG. 8A) aluminum foil, (FIG. 8B) Teflon, and (FIG. 8C) glass.

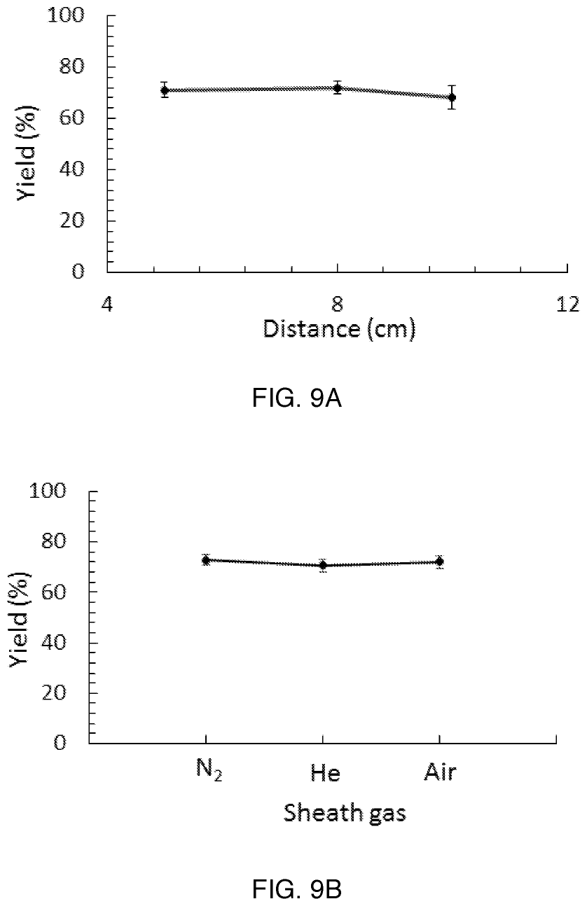

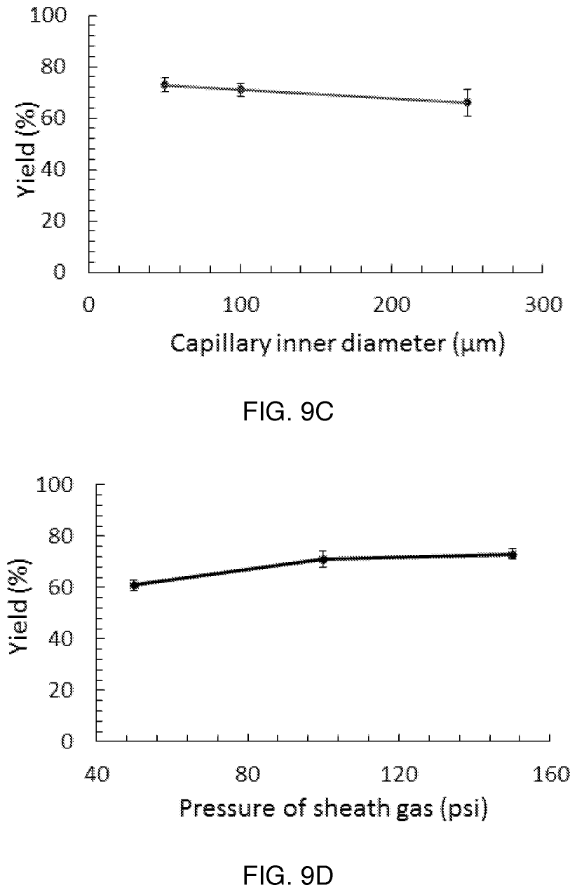

This observation indicated that the reactions were not mediated by the collection surface. The flying distance of microdroplets determined the degree of product conversion in some previous reactions. We changed the distance between the spray emitters and collection surface from 5 cm to 10 cm, and we did not find a product yield change in trend (FIG. 9A), which shows the fast reaction occurred in the microdroplets before landing on the surface. Compressed air and helium gas were also tried as sheath gas with no apparent changes in the yields (FIG. 9B).

Three control experiments were performed in bulk, drop-casted millimeter-size droplets and droplets generated from a 29 nL microT with the reaction time of 10 min or more. GC identified that no reaction occurred in all these three conditions (FIG. 4C). More specifically, FIG. 4C shows the GC spectrum of the two-phase oxidation reaction in bulk, and the other two control cases had similar GC spectra. A previous study also showed that no oxidation occurred in the absence of the phase-transfer catalyst in bulk solution.

The sharp contrast of the two-phase reaction behaviors in microdroplets to bulk and large droplets (100 μm to 5 mm) in the above cases emphasized the importance of droplet size (surface-to-volume ratio) in driving the two-phase reactions. With a decrease of the droplet size from mm to micrometer, the surface-to-volume ratio increases three orders of magnitude. We downsized droplets by either fixing the pressure of sheath gas and using capillaries with inner diameter (i.d.) of 50, 100, and 250 μm and same outer diameter of 360 μm to generate the microdroplets with different initial sizes from each stream (FIG. 9C); or fixing the diameter of the capillary and changing the sheath gas pressure from 50 to 150 psi to decrease the droplet size by increasing the shearing force (FIG. 9D). Slightly increased product formation was observed for a capillary of 50 μm i.d. and under 150 psi gas pressure (FIGS. 9C-9D). This suggests that up to certain level of tiny droplets, further decrease of droplet size has no significant effect on the progression of two-phase reactions in microdroplets.

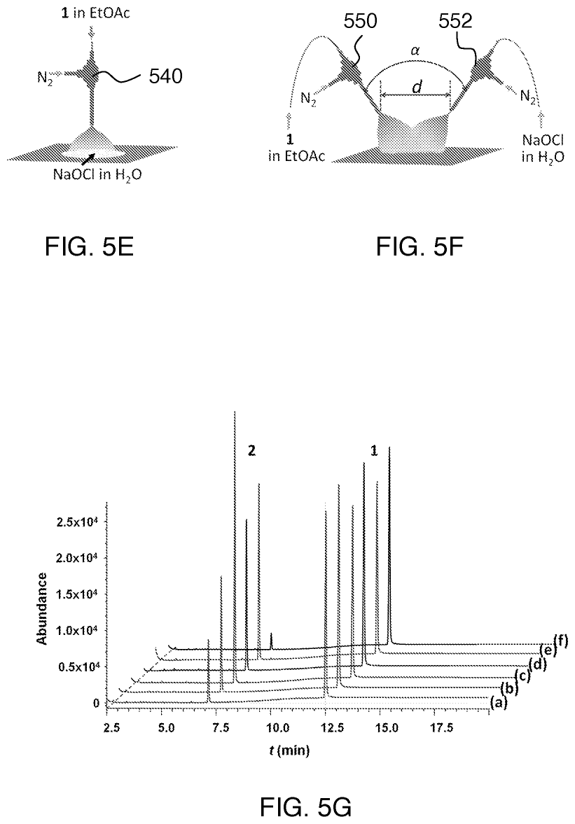

To explore other intrinsic factors that facilitate the liquid-liquid reaction in microdroplets, different methods of generating microdroplets and ways of interacting between the two phase droplets were investigated. FIGS. 5A-5F show various configurations that were tried.

FIG. 5A shows two-phase annular flow that was generated in sprayer 502 by inserting the capillary tube fed with 1 in EtOAC into the capillary tube fed with aqueous NaOCl and nebulized by sheath gas, as shown at 504. The bottom of the inner capillary was first kept at the same level with that of the outer capillary, as shown at 506. The case of FIG. 5B is similar to that of FIG. 5A, except that the inner capillary was set back to the outer concentric capillary, as shown at 516. In the experiment of FIG. 5C two-phase cross flow was formed by mixing 1 and NaOCl in a microT 520 and sprayed with assisted sheath gas by sprayer 522. In the experiment of FIG. 5D microdroplets of 1 in EtOAc was sprayed onto the collection surface followed by spraying NaOCl in water onto the layer of 1 with sprayer 530. In the experiment of FIG. 5E, microdroplets of aqueous NaOCl was sprayed onto the collection surface followed by spraying 1 in EtOAc onto the previous layer of NaOCl with sprayer 540. In the experiment of FIG. 5F a dual spray of 1 in EtOAc and aqueous NaOCl at a certain distance d and angle α was sprayed onto the collection surface with sprayers 550 and 552. FIG. 5G shows GC spectra of two-phase oxidation reaction under the conditions shown in FIGS. 5A-F.

In FIG. 5A, a silica fused capillary tube fed with 1 in EtOAc was inserted inside a concentric capillary tube fed with NaOCl aqueous solution to produce an annular flow. The bottom of the inner capillary was first kept at the same level with that of the outer capillary. Two phases contacted only when they entered the tip of the spray emitter. GC shows that a yield of 18% (FIG. 5G) was obtained. When we set the inner capillary back to the outer concentric capillary (FIG. 5B), a better yield (27%) was resulted. To further increase the contact time of the two phases, we mixed the two phases by cross flow in a microT and kept the droplet segments flowing through a certain length of capillary followed by spaying the droplets to the surface (FIG. 5C). Fairly good conversion from 1 to the product was obtained in some trials. However, the yield varied (from 30%-58%) in different batches. Possible reasons for the unsteady formation of the products might be related to the effect of high pressure sheath gas on mixing two-phase droplets before and after their flowing from the capillary. More studies are ongoing for this device, while a key clue obtained here was NaOCl did not communicate effectively with 1 when it was in the microdroplets in the low yield batches.

To verify this hypothesis, we divided the microdroplet reaction into two steps: microdroplets of 1 in EtOAc was deposited onto the surface followed by spraying aqueous NaOCl onto the layer of 1 (FIG. 5D) or vice versa (FIG. 5E). GC gave repeatable product conversions for both setups, although the yields were relatively low (33% and 38% respectively). This behavior was caused by the fact that the interfacial area of one reagent on the collection surface was not fully used to interact with the other and droplets were partially fused upon their deposition on the surface.

In this regard, we forced the microdroplets of two phases to collide with each other in a Y-shape intersection without touching any other surface before they were collected (FIG. 5F). Note both of the two-phase spray plumes were initiated by sheath gas instead of either one/both by electric field in extractive electrospray or microdroplet fusion experiments. There is a thin intervening gas film between the surfaces of two droplets.

If the collision kinetic energy (majorly gained from sheath gas) of the two droplets is not sufficient to penetrate this gas layer, the droplets bounce off each other, resulting in no physical contact between two liquid droplets. This behavior can be seen from the GC spectrum obtained with the distance between two spray emitters (d) exceeding 80 mm. Almost no product was formed in such microdroplet reactions. The optimized distance d of 1.5 mm with an angle α of 80° between two spray emitters pointing to the surface enabled effective collisions (coalescence, disruption, or/and fragmentation) between microdroplets to occur. A representative GC spectrum obtained under this configuration is shown in FIG. 4B with an overall product yield of 72%.

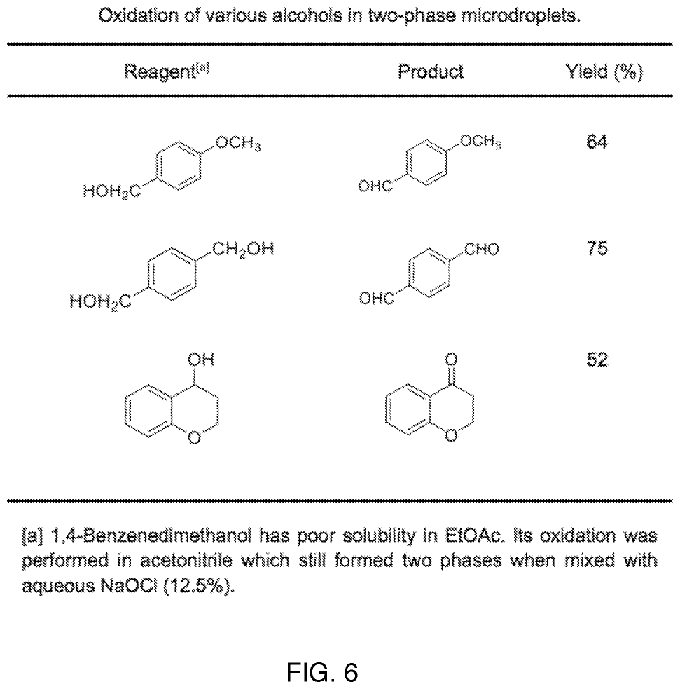

Encouraged by these results, we further examined the microdroplet two-phase oxidations of several other alcohols including benzyl alcohols with different substituents, 1,4-benzenedimethanol and secondary alcohol as shown in the table of FIG. 6. In all cases tested, the desired oxidation products of individual alcohols were obtained without using phase-transfer catalysts in moderate to good yields.

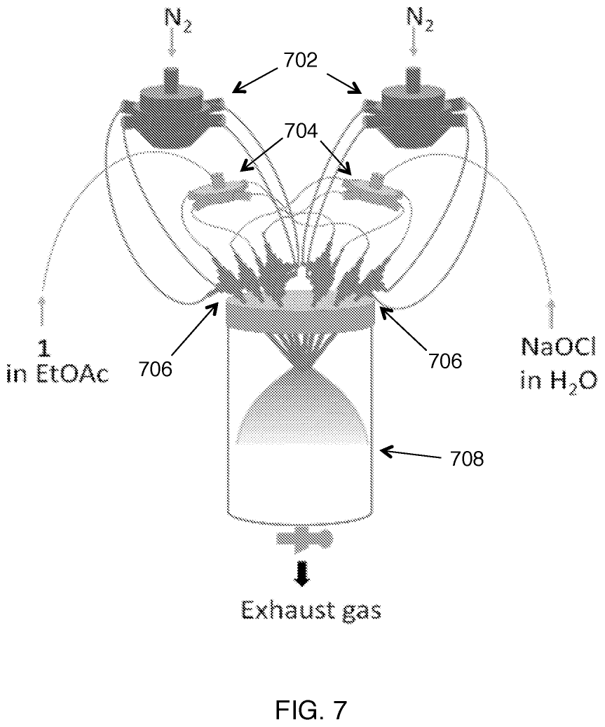

To demonstrate the practical utility of the present two-phase microdroplet synthesis method, a preparative-scale experiment was performed as shown on FIG. 7. This is a scale-up of two-phase microdroplet oxidation of 1 in EtOAc (0.2 M) with aqueous NaOCl (12.5%). Four pairs of dual microdroplet sprayers 706 were arranged in a radial shape and converged at the tips of spray emitters. The two-phase liquids were respectively introduced through the five-port mixers 704 to the spray emitters 706. Sheath gas (N₂) was delivered to the spray emitters 706 using two gas manifold systems 702. Accordingly, a rate of 1.2 mg/min was realized for the synthesis of 4-nitrobenzylaldehyde (2) with the isolated yield of 64% in reaction chamber 708.

In summary, we have demonstrated that two-phase reactions can be carried out in microdroplets rapidly and with good yield without using phase-transfer catalysts. Various alcohols including primary and secondary alcohols were shown to be oxidized to their corresponding aldehydes and ketones. Microdroplets generated by six methods showed different progressions of two-phase reactions. Our results indicate that not only the increased interfacial areas but also effective communications between the microdroplets of two phases play an important role in facilitating the two-phase reactions in the absence of phase-transfer catalysts. A preparative-scale experiment was also performed, and yielded product at an isolated rate of 1.2 mg/min, which demonstrates the possible practical utility of the present method.

### Experimental Section

For two-phase microdroplet synthesis of 4-nitrobenzaldehyde (2), 4-nitrobenzyl alcohol (1, 0.2 M) in ethyl acetate and aqueous sodium hypochlorite (12.5%) were loaded at equal volume into two airtight glass syringes and were delivered with a syringe pump (Harvard Apparatus) at a flow rate of 15 μL/min to two separate capillaries with i.d. of 100 μm, and o.d. of 360 μm. The terminals of the capillaries were equipped with two sheath-gas-assisted spray emitters. The angle between the two spray sources was set between 60° and 80°. The distance between the two capillaries was set in a range of 0.5-2 mm, depending on the angle of the two spray sources. The dry N₂ gas, which served as sheath gas, was operated under 120 psi. Glass surface was used to collect the merged plumes from two spray sources. Upon completion of the reaction, ethyl acetate was used to extract the product from water and the product was dried by sodium sulfite. The yield of product was determined by GC.

### Instrumentation

For GC analysis, samples were run on a Shimadzu GC column with a flow rate of 1 mL/min. Oven temperature was held at 180° C. for 2 min and then increased linearly to 225° C. over 10 min with a final hold of 4 min. GC yields and conversions were determined using standard curves generated from a series of known standards referenced to the internal standard benzaldehyde.

### Section C) Gas-Liquid Reactions

The oxidation of aldehydes to carboxylic acids has been of long-standing interest in synthetic organic chemistry, and is an industrially important process. Popular conventional methods using different oxidizing reagents include Cr(IV)-based Jones oxidation, Ag(I)-based Tollen's reaction, Cu(II)-based Fehling's reaction, permanganate oxidation, periodate oxidation, and Pinnick oxidation. Limitations in these methodologies are quite clear, as they require stoichiometric amounts of highly hazardous oxidants, often take place in harmful solvents, and use sophisticated conditions.

With the growing interest in green chemistry, efficient oxidation processes with environmentally friendly oxidants under mild conditions have become attractive for sustainable chemistry. Molecular oxygen is considered as an ideal oxidant because it is inexpensive, relatively safe for the environment, and it exhibits a highly atom-efficient oxidant per weight (100% atom efficiency). However, methods to achieve direct and efficient oxidation of aldehydes to carboxylic acids using molecular oxygen as the oxidant under mild conditions are still scarce. Most catalytic oxidations of aldehydes with molecular oxygen suffer from the need for rare and expensive noble metals as catalysts, which restricts their use.

Recently, great progress has been made on the development of less expensive transition-metal catalyst systems for oxidations of aldehydes to carboxylic acids.

For example, the Li group reported a homogeneous copper-catalyzed aerobic oxidation of aldehydes in water at 50° C. for 12 hours; the Wei group developed a heterogeneous iron (III)-catalyzed aerobic oxidation of aldehydes in water at 50° C. for 8 hours; and the Favre-Reguillon group found the use of Mn(II) catalyst to be a very efficient for selective aldehyde oxidation. As satisfactory and efficient as the these methods are, however, they still require long reaction times, the use of ligands that are sometimes commercially unavailable or susceptible to oxidative self-degradation, or/and additives that can strongly affect transformation efficacy. We report an alternative approach involving the robust oxidation of aldehydes to carboxylic acids that is performed in microdroplets containing water-ethanol using O₂/air as the sole oxidant under atmospheric pressure with or without catalytic nickel(II) acetate.

It is known that autoxidation of aldehydes into carboxylic acids can occur slowly at the interface when aldehydes are exposed to oxygen or air. Mass transfer across the interface is the rate-controlling step in most of the two-phase reactions. Methods that can increase the interfacial contact area between the two phases should improve mass transfer, resulting in a faster reaction rate and better product conversion. Such methods as the increase of surface interactions in thin layers of aldehydes have been reported. However, the efficiency and yield were poor. The alternative method of using a Rushton turbine or a self-suction turbine for vigorous stirring of bulk aldehyde solution allows accelerated oxidation of aldehydes, while the reactions need to be performed in an autoclave with 8 bar of oxygen or air. “On water” oxidation of aldehydes was carried out by vigorously stirring aldehydes with water in the presence of oxygen, but the reactions are limited to hydrophobic aldehydes, and required extremely sensitive solvent systems where a little amount of organic solvent can completely suppress the reactions.

In the last few years, studies from our group and other groups have shown that solution-phase reactions in microdroplets created by spray-based ionization, surface drop-casting, and microfluidics can be orders of magnitude faster than their conventional bulk-phase counterparts. Recent studies have demonstrated the high surface-to-volume ratio of microdroplets, one of their most prominent features compared with bulk phase, plays an important role in the microdroplet reaction acceleration. The comparison of microdroplet with bulk phase in a study of competitive substituent effects in Claisen-Schmidt reactions showed reagents with more surface activity had more reactivity in the microdroplet. Surface effect has also been observed in atmospheric halogen chemistry, reactions with Criegee intermediates at the air-aqueous interface, and catalytic oxidation of p-xylene to produce high-purity terephthalic acid.

The completion of liquid-liquid phase Stevens oxidations in microdroplets without the use of a phase-transfer-catalyst, as described in section B, provided direct evidence that the increase of interfacial areas in microdroplets drove the reaction at the interface.

In this section a solution of aldehyde dissolved in a water-ethanol mixture was dispersed into microdroplets through a sonic spray source, resulting in largely increased interfacial areas for interactions with molecular oxygen by many orders of magnitude. Molecular oxygen has dual roles of being the oxidant as well as the sheath gas to generate microdroplets. Thus, mixing of two phases occur during microdroplet formation. We demonstrated the methodology in both small-scale synthesis (FIG. 10A) and large-scale preparative synthesis of a carboxylic acid with a modified setup (FIG. 10B). This methodology may serve as a general way of performing fast and efficient gas-liquid two-phase reactions.

FIG. 10A shows two-phase aerobic oxidation of aldehyde into carboxylic acid performed in microdroplets on a small scale in which the microdroplets are generated by the atomization of bulk solution with turbulent nebulizing oxygen gas at 90-120 psi in sprayer 1002. The inset shows a detail view of the sprayer with external mixing of liquid and gas. FIG. 10B shows two-phase aerobic oxidation of aldehyde into carboxylic acid performed in microdroplets on a preparative synthesis scale using modified commercial spray nozzles 1012 and 1014. The inset shows the nozzle with internal mixing of liquid and gas, and a mounted mesh that controls the droplet size.

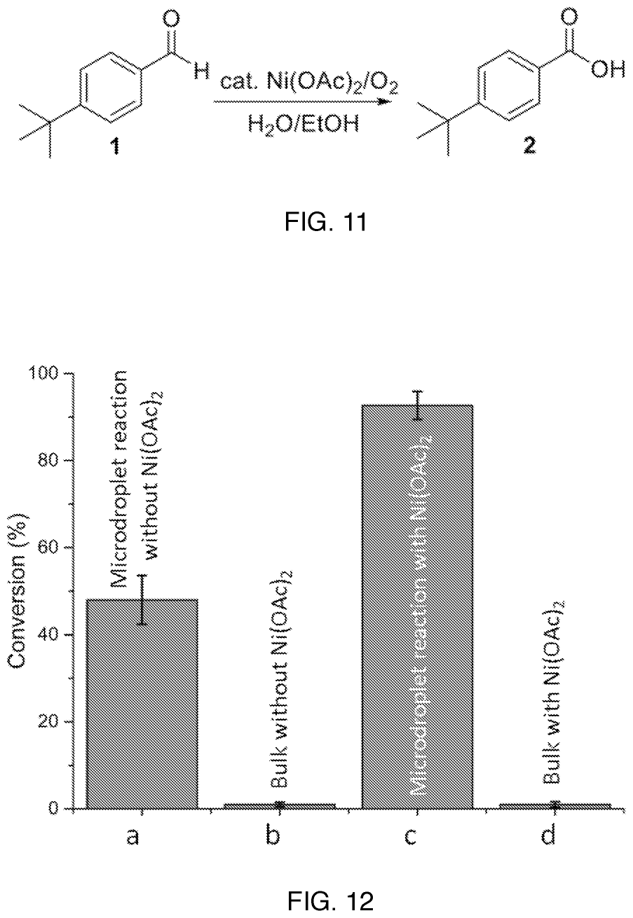

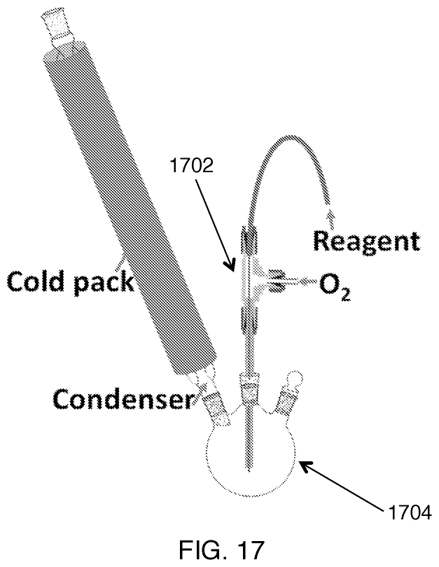

We began our investigation by examining the oxidation of 4-tert-butylbenzaldehyde (1) in a water-ethanol solvent (v:v=1:1.2) with molecular oxygen (O₂) and without any metal catalyst to form 4-tert-butylbenzoic acid (2) according to the scheme of FIG. 11. A water-ethanol solution of 1 (0.1 M) introduced through a fused silica capillary (i.d. 50 μm) at the rate of 15 μL/min was atomized into microdroplets (average size ca. 3.1 μm; see below for method of droplet measurement) with coaxial flow of oxygen being as the turbulent nebulizing gas operated at 120 psi as well as the sole oxidant. The oxidation of 1 was initiated by the interactions between 1 in microdroplets with molecular oxygen at the interface. The resulting products were collected for 30 min using an optimized microdroplet trapping system as shown on FIG. 17. Here sprayer 1702 introduces the reagent droplets and oxygen into chamber 1704, and the resulting products are collected in the chamber 1704. A condenser and cold pack on a gas line were used to prevent loss of volatile compounds.

The reaction mixture was extracted with dichloromethane and analyzed by ¹H NMR. Conversion percentages for various experimental conditions are shown on FIG. 12. Here bar (a) relates to microdroplets without adding Ni(OAc)₂, bar (b) relates to bulk without adding Ni(OAc)₂, bar (c) relates to microdroplets with 5 mol % Ni(OAc)₂, and bar (d) relates to bulk with 5 mol % Ni(OAc)₂. Error bars represent one standard deviation for three measurements.

The conversion of 1 to 2 was found to be 48% (a on FIG. 12). A control experiment was performed in bulk solution (O₂ was supplied in a balloon), and less than 1% of product was detected (b on FIG. 12).

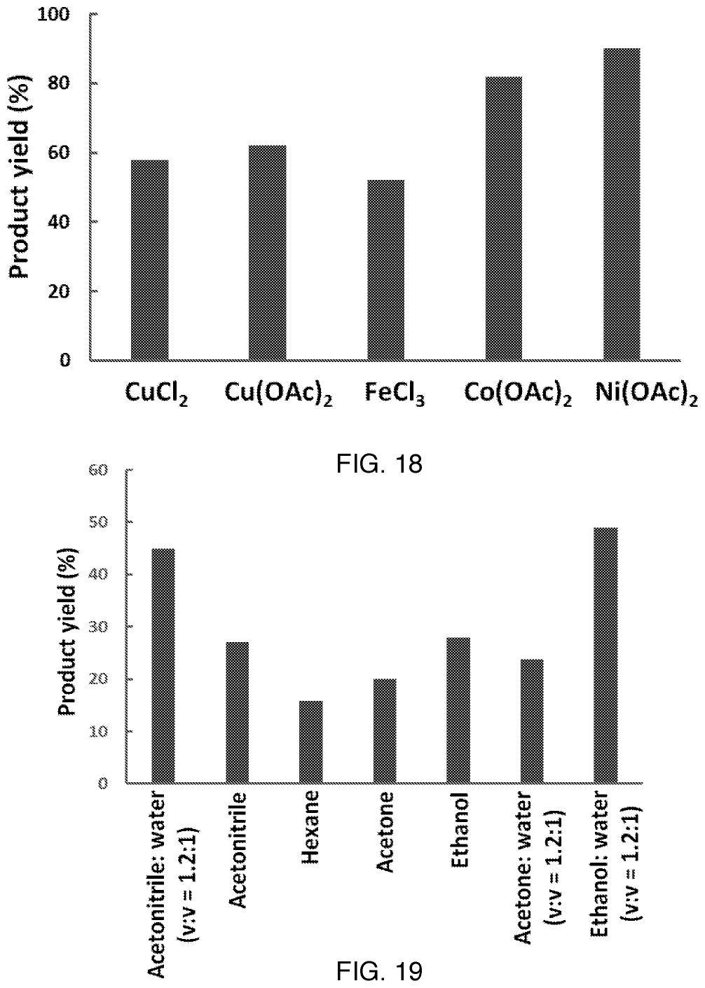

We then screened the widely available and inexpensive metal catalysts without adding any ligand or additive. FIG. 18 shows the screening results. Here oxidation yields of 4-Cert-butylbenzoic acid from 4-tert-butylbenzaldehyde with molecular oxygen in water-ethanol (v:v=1:1.2) in microdroplets catalyzed by 5 mol % CuCl₂, Cu(OAc)₂, FeCl₃, Co(OAc)₂, or Ni(OAc)₂ are shown. A catalytic amount (5 mol %) of nickel(II) acetate showed best efficiency among all the screened catalysts including copper (II) chloride, copper (II) acetate, iron(III) chloride, and cobalt(II) acetate—a conversion efficiency of 91% was achieved (c on FIG. 12). Very low amounts of by-product were observed in the microdroplet reaction compared with bulk reactions under previously reported conditions. Interestingly, the addition of nickel(II) acetate did not catalyze the reaction in bulk (d on FIG. 12).

In addition, the solvent system was investigated because it serves not only as the reaction medium but it also affects the formation of microdroplets. FIG. 19 shows these screening results. Here oxidation yields of 4-tert-butylbenzoic acid from 4-tert-butylbenzaldehyde with molecular oxygen in microdroplets in acetonitrile: water (v:v=1.2:1), acetonitrile, hexane, acetone, ethanol, acetone: water (v:v=1.2:1), or ethanol: water (v:v=1.2:1) are shown. Water-ethanol (v:v=1:1.2) gave the best conversion among various organic solvents as well as miscible aqueous organic solvents.

The liquid-phase oxidation of organic compounds with O₂ as the oxidant can be affected by a complex set of factors which include intrinsic parameters (aldehyde reactivity, solvent, etc.) and extrinsic parameters (catalyst, initiators/inhibitors, etc.), as well as physical phenomenon such as gas to liquid mass transfer. When oxygen transfer becomes the rate limiting step, the rate of the overall process is no longer controlled by the chemical mechanisms but rather by the physical transport phenomena. These considerations and results shown in FIG. 12 (c and d) prompted us to investigate the effect of the surface area-to-volume ratio on the product conversion.

We controlled droplet size by varying the pressure of sheath gas and using capillaries with different inner and outer diameters. Surface-area-to-volume ratio of microdroplets was calculated based on the droplet size measured by micro-particle image velocimetry (μPIV, see below for details). The experiment started with dripping droplets with the surface-area-to-volume ratio of 0.002 through the capillary (i.d. 250 μm, o.d. 365 μm) with no sheath gas supply but in an oxygen environment protected by an O₂ balloon. The flow rate was kept at 15 μL/min, and less than 5% product was formed in 30 min. We increased the surface-area-to-volume ratio of droplets up to 500 times by increasing the O₂ sheath gas pressure from 30 to 120 psi through the capillary (i.d. 50 μm, o.d. 365 μm).

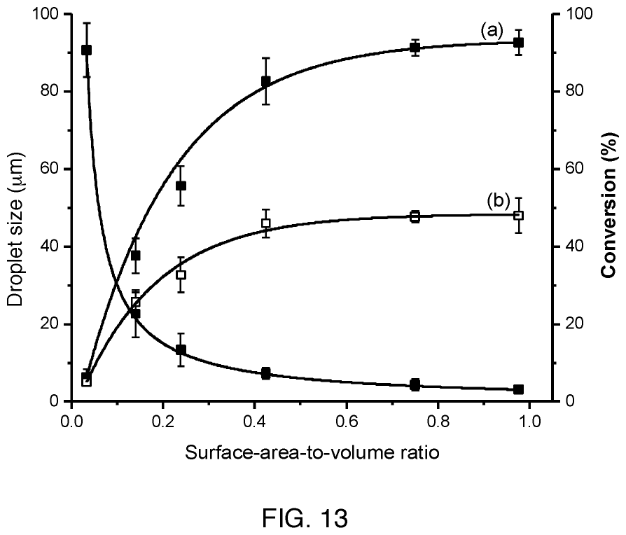

FIG. 13 shows the results of this experiment. More specifically, the surface-area-to-volume ratio dependence of the product conversion in microdroplet aerobic oxidation of 4-tert-butylbenzaldehyde to 4-tert-butyl benzoic acid is shows for two cases: (a) using O₂ as oxidant, and (b) using air as oxidant. The product 4-tert-butylbenzoic acid was largely enhanced with an increase of surface-area-to-volume ratio of droplets from 0.033 to 1 (FIG. 13, a), and reached the maximum conversion when droplet size decreased to about 3 μm. Similar phenomena were also observed using compressed air as the oxidant with less product conversion (FIG. 13, b).

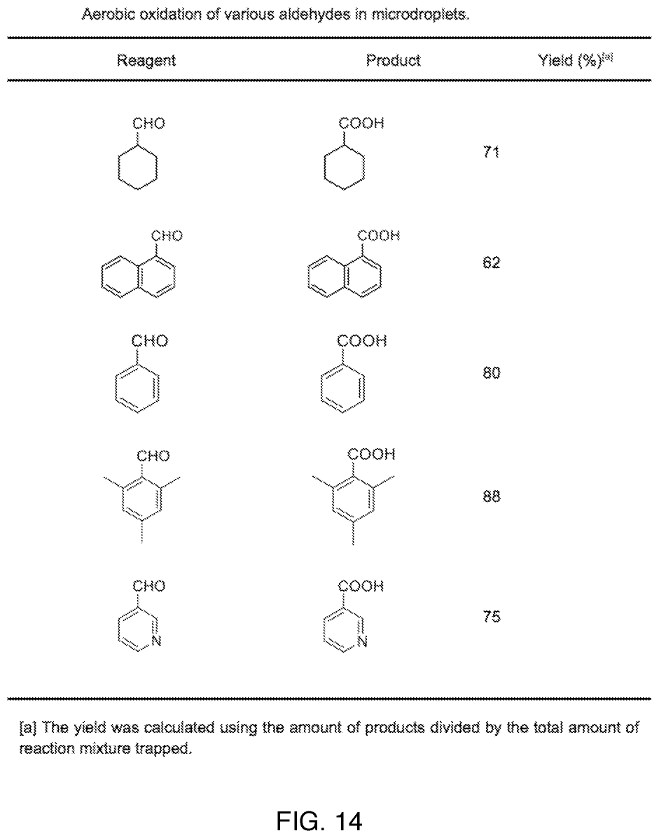

Encouraged by these results, various aldehydes including aliphatic, aromatic and heterocyclic aldehydes were tested under their optimized conditions. The corresponding carboxylic acids were obtained in moderate to good yields as shown in the table of FIG. 14.

The highly efficient and sustainable conversion of aldehydes into carboxylic acids described above inspired us to explore the possibility of scaling up this reaction in microdroplets. Previous studies on “preparative electrospray” employed four or eight spray sources at the same time, and products were generated at rates on the milligram per minute scale for Claisen-Schmidt condensations, benzoin condensations and Stevens oxidations. Further scale up of microdroplet reactions by paralleling more spray sources might not be practical and economical owing to complicated arrangements of splitting gas and liquid, as well as the large demand for duplicated spray sources. Here, we developed a device using two big spray nozzles for fast and large-scale microdroplet synthesis (FIG. 10B).

The regular sprayers (electrospray, sonic spray source, etc.) applied in previous microdroplet work use concentric capillaries (for liquid reagents) inserted into a sheath gas tubing with a length of 1 mm staying outside (FIG. 10A inset). Sheath gas contacts liquid outside the sprayer and shears the liquid into microdroplets. Simply enlarging the capillary size and liquid flow rate from previous spray sources resulted in incomplete atomization of the liquid (especially for the liquid in the middle of the flow), as well as a large distribution of droplet sizes, causing little product (<1%) to be formed. In our design, an internal-mix nozzle (from Unist Co., Grand Rapids, Mich.) was used in which the sheath gas contacts fluid inside the nozzle and disperses it into microdroplets flying throughout the spray hole (FIG. 10B inset). Such a nozzle uses less atomizing gas and generates droplets with a smaller size distribution compared to the previous external mix spray of liquids at the same flow rate. It is also better suited to higher viscosity streams.

The problems with direct use of commercialized internal-mix nozzle for microdroplet reactions are (1) the droplets generated from this nozzle are too large (ca. 90 μm) for accelerated microdroplet reactions (see FIG. 16 below), and (2) increased flow rate (8 mL/min) did not allow 4-tert-butylbenzaldehyde to have a good contact with the oxidant, leading to a reaction yield of less than 5%.

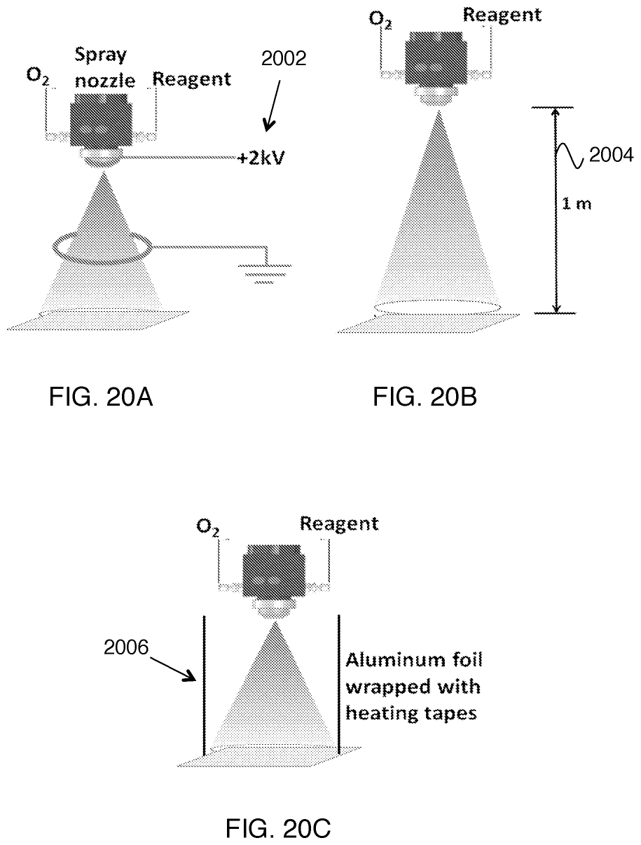

We tried various methods to reduce the droplet size include using electrified droplet fission, and acceleration of droplet desolvation by heating the droplet flying path and extending droplet flying distance. These are schematically shown on FIGS. 20A-C. Here FIG. 20A shows electrified droplet fission caused by applied voltage 2002, FIG. 20B shows acceleration of droplet desolvation by extending droplet flying distance 2004, and FIG. 20C shows acceleration of droplet desolvation by heating the droplet flying path with heater 2006.

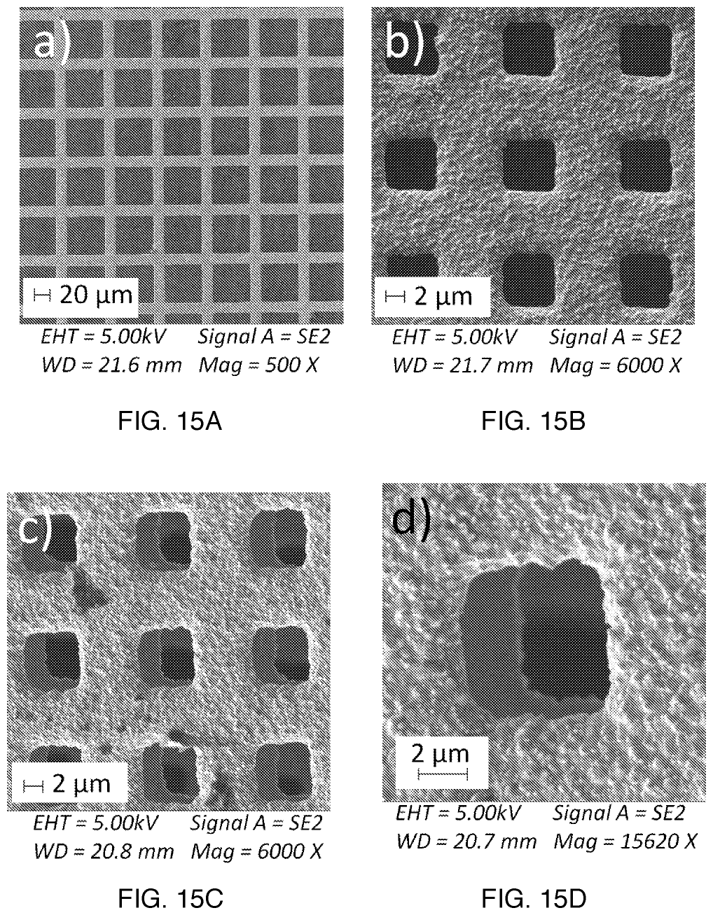

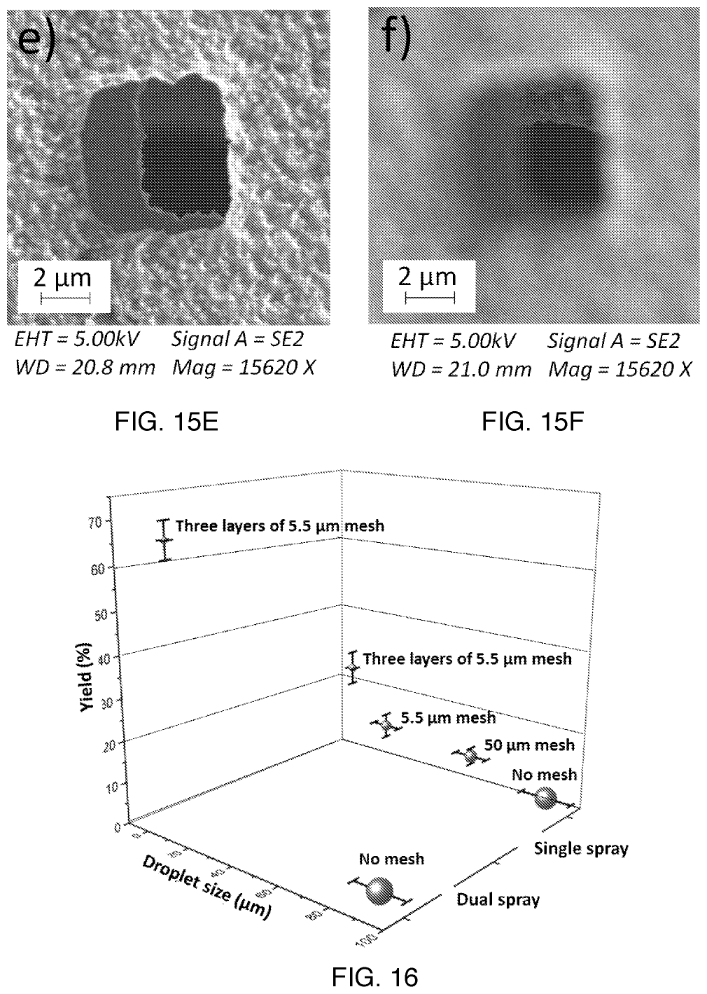

We found that the most efficient method was to mount meshes in front of the spray hole (FIG. 10B). Large droplets were broken into small droplets through size-guided Ni wire meshes. The scanning electron microscopy (SEM) images of FIGS. 15A-F show the meshes of 50 μm, 5.5 μm and three layers of 5.5 μm used in the study. More specifically, FIG. 15A shows a mesh size of 50 μm, FIG. 15B shows a mesh size of 5.5 μm, and FIG. 15C shows a mesh having three layers of 5.5 μm mesh stacked on each other. FIGS. 15D, 15E and 15F are images of the mesh of FIG. 15C with the focus on the first, second and third mesh layers respectively. PIV (particle image velocimetry) was used to measure the sizes of microdroplets generated by internal-mix nozzle mounted with these meshes in a water-ethanol solution.

FIG. 16 shows the size distribution of microdroplets in a mixed solvent of water and ethanol (v:v=1:1.2) generated by internal-mix nozzle after mounting meshes with a size of 50 μm, 5.5 μm, and three layers of 5.5 μm which is plotted against the oxidative conversion of 4-tert-butylbenzaldehyde to 4-tert-butylbenzoic acid under the above conditions. Error bars on the droplet size represent one standard deviation calculated from more than 20 measurements. Error bars on the product yield represent one standard deviation calculated from three measurements. The meshes effectively reduced the droplet sizes, and by overlapping three layers of 5.5 μm mesh (the minimum size we purchased commercially), the droplet size was reduced to about 3 μm, which can be comparable to the size of microdroplets generated in the small sonic sprayer.

Another important factor that allows the reaction to have high conversion yield is the mixing efficacy of gas and microdroplets. In order to increase the interactions between 4-tert-butylbenzaldehyde and O₂, we introduced another stream of O₂ through a similar nozzle but without infusing the liquid. The optimized angle between the two nozzles was set between 60° and 80°. Rapid mixing at the cross section of two fluid streams allows efficient mass transfer between the two phases. Finally, the aerobic oxidation of 4-tert-butylbenzaldehyde to 4-tert-butylbenzoic acid was achieved in a mixture of water and ethanol (v:v=1:1.2) at a product formation rate of 10.5 mg/min with a yield of 66% for the isolated product. As FIG. 16 shows, the highest yield was obtained with small droplets in dual spray.

In summary, we have demonstrated that aerobic oxidation can be carried out in microdroplets much more rapidly and with better yield compared with its bulk-phase counterpart. Addition of catalytic nickel(II) acetate further accelerated microdroplet reaction, while its addition had no apparent effect in the bulk reaction. Aliphatic, aromatic, and heterocyclic aldehydes were oxidized to their corresponding carboxylic acids. O₂ has the dual role of being the sheath gas to generate microdroplets as well as the sole oxidant in the reaction. We also scaled up the microdroplet reactions using the internal-mix nozzle mounted with size-controlled meshes. We achieved a preparative synthesis of 4-tert-butylbenzoic acid with isolated product yield of 10.5 mg/min, which we suggest demonstrates the possible practical utility of the present method.

### Experimental Section

For the small-scale gas-liquid phase microdroplet synthesis of 4-tert-butylbenzoic acid (2), 4-tert-butylbenzaldehyde (1, 0.1 M) and 5 mol % nickel(II) acetate in the water-ethanol mixed solvent (v:v=1:1.2) were loaded into an airtight glass syringe. The solution was delivered with a syringe pump (Harvard Apparatus, Holliston, Mass.) at a flow rate of 15 mL/min to capillaries with an i.d. of 50 μm and o.d. of 360 μm. The end of the capillaries was equipped with sheath-gas-assisted spray emitters. Compressed O₂, which served as the sheath gas and oxidant, was operated at 90-120 psi. Optimized microdroplet trapping system (FIG. 17) was used to collect the plumes from the spray source. Upon completion of the reaction, dichloromethane was used to extract the product from water, and the product was dried by sodium sulfate.

For the preparative synthesis of 4-tert-butylbenzoic acid, 4-tert-butylbenzaldehyde (1, 0.1 M) was dissolved in the water-ethanol mixed solvent (v:v=1:1.2), and pumped through a pipeline from a tank pressurized by nitrogen gas (20 bar) to the spray nozzle. O₂ was operated at 60 psi, and split into two streams: one was introduced to the nozzle housing to mix with the liquid of 4-tert-butylbenzaldehyde and disperse it into microdroplets; the other was supplied for further mixing. Large column (i.d. 15 cm) with a sand core was used to collect the product. Upon completion of the reaction, the product was extracted with dichloromethane and purified by column chromatography with acetyl acetate and hexane (v:v=1:3).

### General Experimental Details

1.1 Chemicals and materials—All chemicals were purchased from Sigma-Aldrich (St. Louis, Mo.) unless otherwise noted. Mesitylene was purchased from TCI (Purchasing-US@TCIchemicals.com). HPLC grade solvents were purchased from Fisher Scientific (Portland, Oreg.). Spray nozzles were purchased from Unist Co., Grand Rapids, Mich. Precision electroformed Ni meshes with sizes of 50 μm, and 8 μm (SEM shows 5.5 μm) were purchased from Precision Eforming LLC. (Cortland, N.Y.). Capillaries were purchased from Polymicro Technologies. Parts to assemble sonic sprayer were ordered from IDEX Health & Science LLC and Swagelok. Syringes were purchased from Fisher Scientific.

1.2 Nuclear magnetic resonance (NMR) spectra were acquired on a Varian Mercury-400 operating at 400 MHz and 100 MHz, and are referenced internally to residual solvent signals. CDCl₃ or D₂O was used as the solvent.

1.3 Scanning electron microscopy (SEM) analyses were performed on a Zeiss Sigma scanning electron microscope with Schottky Field Emission (FE) source and GEMINI electron optical column. A lateral Secondary Electron (SE)

Detector was used. SEM analyses were operated at an accelerating voltage of 5 kV with a working distance of about 20 mm.

### Measurement of Droplet Sizes

Micro-particle image velocimetry (μPIV) was used to measure the droplet sizes in the study. The method is similar to that reported for imaging the electrospray plume (E. T. Jansson, Y.-H. Lai, J. G. Santiago, R. N. Zare, J. Am. Chem. Soc. 2017, 139, 6851-6854, hereby incorporated by reference in its entirety). Briefly, the determination of droplet sizes was done by elastic light scattering using pulsed 2^{nd} harmonic Nd:YAG lasers (λ=532 nm) plus additional optics. An objective (5× magnification, NA=0.15) was used to gather light and produce imaging onto an interline-transfer CCD camera with a double-frames imaging feature. In this method, the imaging recorded by CCD is the convolution of the point response function, which depends on the optics and illumination wavelength, and the actual droplet size. The droplet size is calculated based on the average number of pixels that droplets occupy on the imaging plane. Surface area-to-volume ratio of microdroplets is derived from the droplet size. It should be noticed that the actual droplet size less than 1.3 μm in diameter will be recognized as a droplet of about 1.3 μm owing to the point response function.

1. A method for performing a chemical reaction, the method comprising:
selecting a first liquid reagent;
selecting a second liquid reagent, wherein the first liquid reagent and the second liquid reagent are immiscible with respect to each other;
nebulizing the first liquid reagent in a first shearing gas flow to provide first microdroplets of the first reagent;
configuring the second liquid reagent as second microdroplets of the second reagent in a second shearing gas flow, or as a thin film of the second reagent;
directing the first microdroplets at the second reagent to provide a chemical reaction between the first and second liquid reagents by colliding the first microdroplets with the second reagent.

2. The method of claim 1, wherein the first microdroplets have a diameter from 0.1 micron to 100 microns.

3. The method of claim 1, wherein the second microdroplets have a diameter from 0.1 micron to 100 microns or the thin film of the second reagent has a thickness of 100 microns or less.

4. The method of claim 1, wherein the chemical reaction is selected from the group consisting of: C-, N-, O- and S-alkylation; etherification; esterification; transesterification; condensation; carbene reaction; nucleophilic displacement epoxidation; oxidation; and polymerization.

5. The method of claim 1, wherein a reaction time of the chemical reaction is 1 second or less.

6. A method for performing a chemical reaction, the method comprising:
selecting a liquid reagent;
selecting a gaseous reagent;
nebulizing the liquid reagent in a first shearing gas flow to provide microdroplets of the liquid reagent;
directing the microdroplets at the gaseous reagent to provide a chemical reaction between the liquid reagent and the gaseous reagent by colliding the first microdroplets with the gaseous reagent.

7. The method of claim 6, wherein the first shearing gas flow is a shearing gas flow of the gaseous reagent.

8. The method of claim 6, wherein the gaseous reagent is provided at least in part in a second gas flow distinct from the first shearing gas flow, and wherein the first shearing gas flow and the second gas flow are directed at each other.

9. The method of claim 8, wherein the first shearing gas flow is a shearing gas flow of the gaseous reagent.

10. The method of claim 6, wherein the microdroplets have a diameter from 0.1 micron to 100 microns.

11. The method of claim 6, wherein the chemical reaction is selected from the group consisting of: C-, N-, O- and S-alkylation; etherification; esterification; transesterification; condensation; carbene reaction; nucleophilic displacement epoxidation; oxidation; and polymerization.

12. The method of claim 6, wherein a reaction time of the chemical reaction is 1 second or less.
